(19) United States
(12) Patent Application Publication
(10) Pub. No.: US 2013/0035606 A1
(43) Pub. Date: Feb. 7, 2013

(54) Multi-Wave Signals to Reduce Effects of Electrode Variability

(71) Applicant: Brian D. Wichner, Otter Rock, OR (US)

(72) Inventor: Brian D. Wichner, Otter Rock, OR (US)

(21) Appl. No.: 13/648,247

(22) Filed: Oct. 9, 2012

(51) Int. Cl. A61B 5/0488; A61B 5/053

(52) U.S. Cl. 600/546; 600/547

(57) ABSTRACT

Subject matter includes a method comprising: applying a first electrical signal to particular locations of a subject via at least one electrode pair; applying a second electrical signal to the particular locations of the subject via the at least one electrode pair, wherein the second electrical signal is applied alternately and repeatedly with the first electrical signal; determining a first electrical impedance based, at least in part, on the first electrical signal; determining a second electrical impedance based, at least in part, on the second electrical signal; and removing at least a portion of varying electrical characteristics of the at least one electrode pair by calculating a difference between the first electrical impedance and the second electrical impedance.

### BACKGROUND

1. Field

Subject matter disclosed herein relates to detection of one or more physical conditions of a subject by applying electrical energy to the subject, and more particularly to reducing influence of electrode variability on such detection.

2. Information

A number of techniques for treating a subject (e.g., a patient) or detecting a physical condition of a subject may involve applying electrical energy via electrodes in contact with the subject. Such electrodes may comprise pads having an adhesive (or a water-activated adhesive) to temporarily affix the pads to a portion of a subject. For example, a transcutaneous electrical nerve stimulation (TENS) device may apply electric current to a subject via electrode pads to stimulate nerves of the subject for therapeutic purposes. In another example, muscle loss of a subject may be determined using electric impedance myography (EIM), which may measure resistance of a muscle to an electrical current by passing an amount of current through the muscle using two electrodes.

Electrical current of electrodes applied to a subject may flow through a number of characteristic regions of the subject. For example, current from a first electrode applied to skin may flow through the skin and subsequently, in varying degrees, through plasma, fascia, muscle tissue, bones, ligaments, and/or organs, and out through skin to a second electrode. Such individual characteristic regions have particular electrical properties, such as electrical resistance, impedance, capacitance, and so on. Undesirably, electrical properties of a skin-electrode pad interface may vary in response to changes in contact area and/or pressure of the electrode pad against the skin, effectiveness of an adhesive to hold the pad to the skin, localized skin and/or pad conductivities, and so on. Such variable electrical properties of a skin-electrode pad interface may be further accentuated in a situation where a subject is in motion. Variable electrical properties may also occur for a skin-electrode interface, wherein electrodes need not comprise pads, but may also comprise needles (e.g., transcutaneous contact), straps, belts, non-contact proximity probes, and so on.

### BRIEF DESCRIPTION OF THE FIGURES

Non-limiting and non-exhaustive embodiments will be described with reference to the following figures, wherein like reference numerals refer to like parts throughout the various figures unless otherwise specified.

FIG. 1 is a cross-sectional schematic diagram illustrating electrodes for applying one or more electrical signals to a portion of a subject, according to an embodiment.

FIG. 2 is a cross-sectional schematic diagram illustrating various paths traveled by one or more electrical signals in a portion of a subject, according to an embodiment.

FIG. 3 is a cross-sectional schematic diagram illustrating an electrical component analogue corresponding to a portion of a subject, according to an embodiment.

FIG. 4 is a cross-sectional schematic diagram illustrating an electrical component analogue corresponding to a portion of a subject, according to another embodiment.

FIG. 5 is a plot of characteristics for relative resistance of a portion of a subject for two electrical signals as a function of time, according to an embodiment.

FIG. 6 includes a number of example waves plotted as magnitude of voltage or current versus time, according to an embodiment.

FIG. 7 is a flow diagram of a process for determining a physical condition of a subject, according to an embodiment.

FIG. 8 is a schematic block diagram illustrating a system for performing a process for determining a physical condition of a subject, according to an embodiment.

FIGS. 9A and 9B are plots of characteristics for first and second electrical signals as a function of time, according to an embodiment.

FIG. 10 is a schematic block diagram illustrating a system and various functions, according to an embodiment.

### DETAILED DESCRIPTION

Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments.

Impedance may refer to the opposition that a path of electrical current presents to the passage of the current if a voltage is applied. For example, in quantitative terms, impedance may comprise a complex ratio of the voltage to the current. Impedance (e.g., for time-varying electrical signals) may comprise an extension of the concept of resistance (e.g., non-time-varying electrical signals), and may include both magnitude and phase, unlike resistance, which may only include magnitude. In situations involving time-varying electrical signals, mechanisms in addition to normal resistance (e.g., ohmic resistance for non-time-varying electrical signals) may impede flow of current. Such mechanisms may comprise induction of voltages in conductors self-induced by magnetic fields of currents (inductance), and electrostatic storage of charge induced by voltages between conductors (capacitance). Impedance based, at least in part, on these two effects may collectively be referred to as reactance and forms an imaginary part of complex impedance whereas resistance forms a real part, for example.

The terms “resistance” and “impedance” are used herein interchangeably to mean the same thing unless used in the context of a sentence that indicates otherwise. For example, “resistance” means impedance that may comprise an inductive reactance, capacitive reactance, and/or ohmic resistance. On the other hand, “impedance” may mean ohmic resistance and may or may not include inductive reactance and/or capacitive reactance. Again, a context or description of a sentence or portion of text in which such terms are used may indicate one meaning over another meaning. The term “resistance” may comprise inductive reactance, capacitive reactance, and/or ohmic resistance. If “resistance” is intended to exclude inductive reactance and/or capacitive reactance then the term “ohmic resistance” is used.

The term “subject” is recited in examples herein. Unless otherwise described, a subject may comprise human, animal, fish, reptile, bird, and so on. A subject may also comprise abiotic systems or material, such as liquid, mineral, plastics, etc., although example embodiments are directed to biotic systems. For example, embodiments of techniques described may be applied in cases where a subject is human or where a subject is a fish or animal, and claimed subject matter is not limited in this respect. To describe a particular implementation, techniques may be applied to diagnose a physical condition (e.g., muscle mass, cancer, blood chemistry, and so on) of a human subject. In another implementation, techniques may be applied to perform research regarding any of a number of physical parameters of various aquatic species. In the latter implementation, the “subject” may comprise a particular aquatic specimen. Other implementation may involve animals, and so on. Accordingly, though the following descriptions may indicate a human subject, claimed subject matter is not limited in this respect.

Biological elements of a subject may comprise any portion or combination of portions of the subject, such as skin, muscle tissue, organs, normal or cancer cells, blood, ligaments, tendons, bones, scar tissue, and so on. Such biological elements may be microscopic or macroscopic. Such biological elements may be in any type of condition, such as healthy or normal, damaged or injured, deteriorated, inflamed, and so on.

In some embodiments, applications of electrical energy (e.g. for muscle stimulation, physical diagnosis, and so on) may involve a power source, a signal generator, at least two electrodes, and leads (e.g., cables, wires, conductors, and so on). Electrical energy application may comprise transcutaneous application, involving leads or electrodes on skin of a subject. Penetration of electrical energy applied to skin may depend, at least in part, on resistance of the skin and/or electrode-skin interface, which may change over time for any number of reasons. For example, during application of electrical energy, skin may become slightly more hydrated under electrodes so that skin resistance may decrease. Also, if an electrode's adhesion to skin changes, the resistance of an electrode-skin interface may also change. For example, if an electrode, or a portion thereof, is pushed against skin, resistance may decrease. On the other hand, if an electrode, or a portion thereof, is pulled or lifted away from skin, resistance may increase. Such pulling or lifting may occur, for example, if a subject is moving so as to flex the shape of the skin surface (e.g., skin may be more pliable than an electrode pad). In such a case, electrical contact between electrode and skin may increase and decrease somewhat cyclically if a subject if engaged in repetitive motion (e.g., during muscle training, conditioning, or exercises). Such pulling or lifting may also occur, for example, if an adhesive begins to weaken over time or because of skin sweating, and so on. Changing electrical resistance between electrode and skin (e.g., an electrode-skin interface) may be undesirable in that electrical current of electrical energy applied to a subject may correspondingly change. Such changes or variability may bring about a degree of undesirable variability, error, or uncertainty of measurements of parameters associated with applied electrical energy. For example, such parameters, which may comprise electrical voltage, current, phase shift, and so on, may be used to determine physical conditions of a subject, such as muscle mass, bone density, organ disposition, and so on.

In an embodiment, a method or technique may reduce or substantially eliminate effects of variability in electrode-skin interface resistance on measurements of parameters associated with applied electrical energy. In an implementation, a system or device may perform such a method. Such a method or technique may comprise measuring a first electrical resistance based, at least in part, on a first electrical signal applied to a subject via one or more electrode pairs. The method may further comprise measuring a second electrical resistance based, at least in part, on a second electrical signal applied to the subject via the same one or more electrode pairs as for the first electrical signal. The second electrical signal may be applied repeatedly and alternately with the first electrical signal or simultaneously with the first electrical signal. For example, a frequency (e.g., or a set of Fourier frequencies) and/or waveshape of a first electrical signal may be substantially different (e.g., different by more than one or two percent of each other) from a frequency and/or waveshape of a second electrical signal.

In another example, a peak voltage of a first electrical signal may be substantially the same (e.g., within a few percent of each other) as a peak voltage of a second electrical signal. In an implementation, the electrodes may be maintained in the same locations on the subject during application of the first and second electrical signals. In other words, any movement of the electrodes with respect to a subject (e.g., slipping across the skin, peeling away from the skin, and so on) may be inadvertent, while it may be desirable to maintain the electrodes in their locations by (temporarily) attaching (e.g., straps, adhesive, and so on) to the subject.

An electrode pair used to apply a signal to a subject may comprise a first electrode and a second electrode. The first electrode may comprise a “+” electrode and the second electrode may comprise a “−” electrode, though the symbols “+” and “−” need not indicate positive or negative portions of the signal. In one implementation, such symbols may indicate polarity of one electrode relative to the other electrode. In another implementation, such symbols may indicate an anode for the positive electrode and a cathode for the negative electrode.

In one implementation, a first electrical signal and a second electrical signal may be applied simultaneously to a subject via an electrode pair (e.g. “+” and “−” electrodes adhered to a subject's skin). In such a case, for example, voltages of two electrical signals may be superposed and the corresponding current may be decomposed to separate the signals so that resistance values of the individual signals may be measured, as described below.

In another implementation, a first electrical signal and a second electrical signal may be applied at different times to a subject via an electrode pair. For example, a first electrical signal and a second electrical signal may be alternately applied to a subject via an electrode pair (e.g., apply a first signal for one second, apply a second signal for the subsequent second, apply the first signal again for the next second, apply the second signal again for the next second, and so on).

The method may further comprise removing varying electrical characteristics of the one or more electrode pairs by calculating a difference between the first measured electrical resistance and the second measured electrical resistance. In one implementation, a physical condition of a subject may be based, at least in part, on such a difference between the first measured electrical resistance and the second measured electrical resistance. For example, a physical condition of a subject may comprise blood chemistry, bone density, cancer presence, or muscle condition (e.g., density, atrophy, damage, imbalance, etc.).

In one implementation, a first electrical signal and a second electrical signal may be applied to a subject while the subject is substantially moving (e.g., movement greater than a few millimeters or centimeters). Such movement may comprise displacement or rotation of the whole subject, or portions thereof. For example, a human subject may be engaged in repetitive or non-repetitive exercises (e.g., pull-ups, jumps, sit-ups, and so on) or activities (walking, running, swimming, and so on) while electrical energy (e.g., first and second electrical signals) is applied via electrodes to the subject. In another example, because of an inherent nature (e.g., untrained, in a wild setting, non-sedated, and so on) of a non-human subject (e.g., animal, fish, etc.), such a subject may be moving while electrical energy is applied via electrodes.

In another implementation, a first electrical signal and a second electrical signal may be applied to a subject while at least one electrode is submerged in a liquid. For example, electrical energy may be applied to a portion of a subject that is submerged in a water bath. In some implementations of such a situation, at least a portion of a water bath may be considered to comprise an electrode. For example, a foot or hand of a subject may be submerged in a water bath so as to treat the foot or hand, though claimed subject matter is not so limited.

In a particular implementation, one electrode may be replaced by two or more electrodes having a combined area similar to the one being replaced. Water bath application of electrical current may involve a hand, forearm, leg or foot being immersed in water through which electrical current is flowing. Bare metal or carbon rubber electrodes may be placed within a water bath (salt may not be used because it may facilitate current flow through the water, reducing current through tissue of the subject). Water may provide a sufficiently conductive ionic medium so that there may be little risk of uneven applications of current to a hand, forearm, leg, or foot.

In another implementation, an apparatus may comprise a port to provide an output signal to a subject via at least one electrode pair. The apparatus may further comprise a circuit to generate the output signal comprising a first electrical signal and a second electrical signal. Such a circuit, which may comprise discrete electronic components and/or a processor, may further determine a first electrical resistance based, at least in part, on the first electrical signal and a second electrical resistance based, at least in part, on the second electrical signal. The circuit may remove at least a portion of varying electrical characteristics of the at least one electrode pair by calculating a difference between the first electrical resistance and the second electrical resistance. For example, varying electrical characteristics of an electrode may comprise varying resistance of the electrode-skin interface, as described below.

In an implementation, a first electrical signal and a second electrical signal may repeatedly alternate between one another in an output signal. However, in another implementation, a first electrical signal and a second electrical signal may be simultaneously present in an output signal. In the latter case, the circuit may be further adapted to measure a composite electrical current responsive, at least in part, to the output signal and electrical properties (e.g., impedance) of the subject. The composite electrical current may be decomposed to determine a first current and a second current responsive, at least in part, to the first and second electrical signals, respectively. For example, the circuit may perform signal decomposition by Fourier transforming a composite signal to obtain component signals, though claimed subject matter is not limited in this respect. Accordingly, first and second electrical resistances may be determined or calculated based, at least in part, on the first and second currents, respectively.

In yet another implementation, if a difference between a first electrical resistance of a first signal and a second electrical resistance of a second signal is less than a particular threshold for a particular subject, then the frequency of the first electrical signal or the second electrical signal may be changed so as to increase the difference. For example, while attempting to measure a property of muscle tissue, a difference between a first electrical resistance of a first signal and a second electrical resistance of a second signal applied to the subject may be less than a particular threshold. In such a case, properties of the first and/or second signal may be inadequate for such a measurement for any of a number of reasons. For example, a frequency of the first signal may be outside a frequency range for which the first signal can reach into the muscle tissue. Accordingly, a difference between the first electrical resistance of the first signal and the second electrical resistance of the second signal may be less than a threshold, which may indicate an inadequate condition for measuring the muscle tissue. Accordingly, the frequency of the first signal (e.g., and/or the second signal) may be increased (e.g., or decreased) until the resistance difference increases. Here, the increasing frequency of the first signal may lead to increasing penetration into the muscle tissue that is to be measured. Such frequency changes may be performed automatically in response to feedback of measure resistances to a processor, for example.

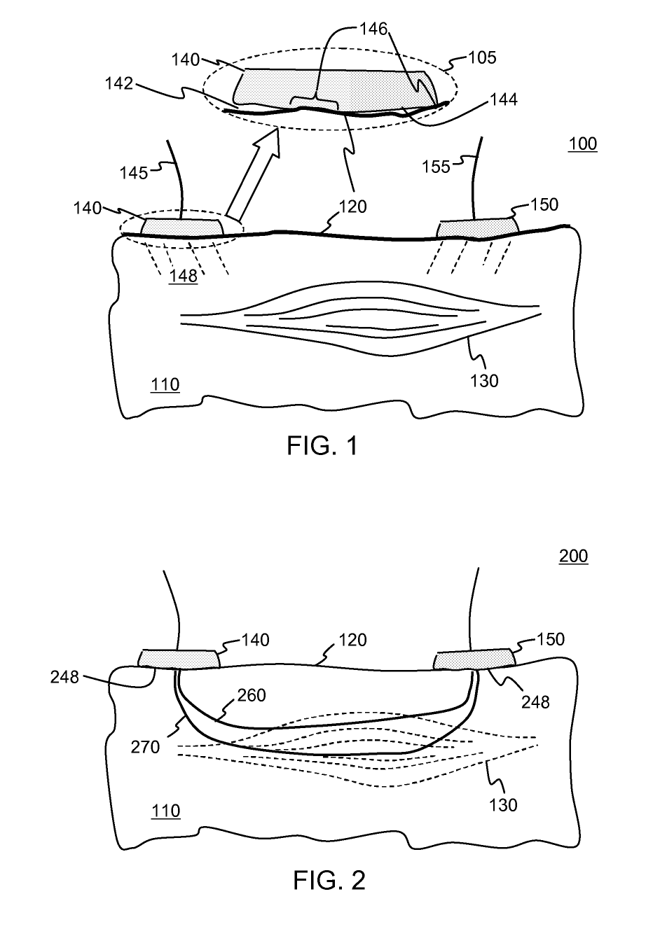

FIG. 1 is a cross-sectional schematic diagram illustrating electrodes 140 and 150 for applying or distributing one or more electrical signals to a portion 110 of a subject, according to an embodiment 100. Portion 110 may comprise a volume of body mass including skin 120 and muscle 130. For sake of clarity, portion 110 may include other biological elements or material that which are not shown. For example, such biological elements or material may comprise DNA, normal or cancer cells, fascia, bone, ligaments, organs, plasma, blood vessels, arteries, and so on. Leads 145 and 155 may carry electrical signals to/from electrodes 140 and 150, respectively. A general flow of electrical signals is schematically indicated by symbol 148. Electrodes 140 and 150 may comprise a self-adhesive, metal foil, or conductive rubber (e.g., carbon-impregnated silicone rubber) electrode. In some implementations, a coupling medium may be used to provide a conductive bridge between the electrode and the skin, such as by filling in voids or gaps (e.g., 142), or by increasing conductivity of skin or electrode surfaces. A coupling medium may be an integral part of self-adhesive electrodes, for example. With conductive rubber electrodes an adhesive gel pad may be used. A coupling gel-pad, which may be solid but soft and flexible, may be both electrically conductive and adhesive. Electrodes may also be strapped onto skin, with or without a coupling medium. A coupling medium for metal foil electrodes may comprise an electrode gel or a wetted pad of lint, cotton gauze, or some form of sponge material that absorbs and retains water, for example. Metal electrodes using spread-able gel or wetted pads may be held in contact to skin by straps or bandages.

Electrical resistance between an electrode and skin (e.g., an electrode-skin interface) may vary for a number of reasons. Actual electrical contact area between an electrode and skin may be responsive to a number of variables, such as skin condition (e.g., smooth, rough, hairy, and so on), method of electrode attachment (e.g., adhesive, elastic bands or straps, and so on), applied forces on electrode, just to list several examples. Moreover, electrical contact area between an electrode and skin may vary over time for any of a number of reasons, such as a changing force applied to an electrode against skin, motion of a subject, changing strength of adhesive holding the electrode to skin, and so on. Accordingly, electrical resistance between an electrode and skin may vary over the course of seconds, minutes, or hours, for example.

A close-up view 105 shows some detail where voids 142 may exist between a bottom surface of an electrode, such as 140, and skin 120. For example, merely a fraction of a surface area of an electrode may physically contact skin while other portions of the surface area of the electrode may be elevated above the skin. Portions 146, for example, may be in physical contact with skin 120. Accordingly, while electrical signals may readily travel between skin 120 and electrode 140 in such contact areas, voids 142 may present resistance to electrical flow. Water or conductive gel may be used to fill voids 142 to improve overall electrical conduction (e.g., reduce electrical resistance) between skin and electrode 140.

Proportion of void area to physical contact area between an electrode and skin may change if the contour of the skin changes relative to that of the electrode. Such relative contour changes may occur if a subject is moving, for example. Change of force of application of an electrode against skin may also change proportion of void area to physical contact area. For example, if an electrode is pushed against skin then void area may be reduced, whereas if an electrode is lifted away from skin then void area may be increased. Electrical resistance between electrode and skin may vary accordingly: pushing an electrode to skin may reduce resistance, whereas forces that tend to lift an electrode away from skin may increase resistance.

FIG. 2 is a cross-sectional schematic diagram illustrating various paths traveled by one or more electrical signals in the portion 110 of a subject introduced above, according to an embodiment 200. An amount of current flowing through tissue may depend, at least in part, on applied voltage between electrodes 140 and 150 and resistance, according to Ohm's Law, V=IR, for example. Here, V is applied voltage, R is resistance and I is current flow. Current of an electrical signal may flow from one electrode to an opposite electrode along any of a number of particular paths 260 and 270 in a subject. As explained below, such paths may depend, at least in part, on electrical and/or chemical properties of internal portions of a subject, such as plasma, muscle, organs, cell structure, just to name a few examples. Also as explained below, a path traveled by an electrical signal may depend, at least in part, on the voltage of the signal and/or the frequency of the signal, among other things. Though not shown, paths 260 and 270 may include any of a number or variety of biological elements (e.g., bone, organs, plasma, cells, tumors, various types of biological tissue, and so on), in addition to muscle tissue 130 shown in FIG. 2.

For example, current of an electrical signal may flow from electrode 140, through couplant 248 (e.g., water or gel), skin 120, into underlying tissues (e.g., 130), and then through another layer of skin and couplant to electrode 150. A total resistance of such a path may comprise a sum of the resistances in each part of the current pathway if the parts are in series (e.g., meaning that current may flow through each part in turn). In an implementation, resistance of electrodes and couplant (which need not be present) may be relatively low. Moreover, resistance of subcutaneous tissue, which may be highly hydrated, may also have a relatively low resistance. Skin resistance, however, may be much higher due, at least in part, to a relatively high resistance of the stratum corneum of skin. In more detail, skin may comprise the dermis and epidermis. The epidermis may be punctured by various skin appendages, such as sweat gland ducts and hair follicles. Beneath skin is the subcutis, also referred to as superficial fascia or simply subcutaneous tissue. In most areas of a subject, the subcutis may be predominantly adipose (fat storing) tissue. Blood vessels, lymph vessels and nerves may infiltrate the subcutis and dermis, but not the epidermis. The dermis and subcutis have relatively low electrical resistance. The subcutis, which may be adipose tissue, may have a relatively low resistance, even though fat is an insulator. The low resistance may be due, in part, to conductive channels (blood and lymph vessels) that may infiltrate the subcutis tissue.

Blood or lymph vessels may not infiltrate the epidermis, which may be avascular, meaning that these cells (e.g., keratinocytes) derive their nutrients by diffusion from capillaries in underlying dermis. A basal layer of the epidermis may be metabolically very active, with the cells regularly undergoing mitosis. Keratinocytes, formed and pushed upwards from this layer, may synthesize keratin, which may be retained within the individual cells. In their life cycle, the keratinocytes may move toward the skin surface, becoming less metabolically active as diffusion limits the rate of nutrient supply. Near the surface the cells may die and shrivel, finally forming a scaly shell called the stratum corneum. The stratum corneum may thus comprise a layer of shriveled, dead, dehydrated keratinocytes, which may further comprise packages of keratin. This structure may contribute significantly to the relatively high resistance of skin.

As mentioned above, an electrical signal may follow a path depending, at least in part, on electrical and/or chemical properties of internal portions of a subject. For example, electrical conductivity of muscle may be different from that of bone or a particular organ. Moreover, as an example, electrical conductivity of muscle tissue or bone may depend, at least in part, on the health or density of the muscle tissue or bone (or portion thereof). In the case of muscle tissue, for example, measurements of electrical conductivity of muscle tissue may be used to determine muscle loss or gain in subjects with Lou Gehrig's Disease, also known as amyotrophic lateral sclerosis, or ALS. This disease may attack motor neurons that control voluntary muscle movement, leading to muscle weakness and atrophy. As ALS spreads, motor neurons may die off, causing muscles to atrophy. Deteriorating muscles may behave differently from healthy ones, resisting electrical current more, for example. Such variations in behavior may be correlated with disease progression and length of survival of a subject. As another example, electrical conductivity of internal portions of a subject may depend, at least in part, on tissue density, presence of cancer cells, and so on.

Also mentioned above, a path traveled by an electrical signal may depend, at least in part, on the voltage and/or the frequency of a signal applied to a subject via electrodes. For example, a relatively low frequency signal, such as below 10,000 Hz may travel through connective biological tissue, but not through individual cells. As the frequency increases above 10,000 Hz, a signal may begin to penetrate outside layers of cells. Above 100,000 Hz, cell penetration may be substantial. In another example, organ tissue density may vary from organ to organ. As tissue density increases, so does electrical resistance to relatively low frequency signals (e.g., below 10,000 Hz). Accordingly, an electrical signal having one frequency may follow a path different from a path followed by an electrical signal having another frequency.

In examples above, the term “resistance” is used. However, as noted earlier, “impedance” may further describe the case of an electrical signal traveling through internal portions of a subject, particularly if such an electrical signal includes a non-zero frequency or phase. Different internal portions of a subject may have different resistivities and/or different capacitances. Examples above touched on ideas that different biological elements may have different resistivities, which may affect current or voltage of a signal. Moreover, different biological elements may have different capacitances, which may affect current, voltage, or phase of a signal. For example, a time-varying (e.g., a sinusoid) electrical signal may experience a shift in phase between current and voltage based, at least in part, on integrity of muscle tissue (e.g., tissue density, mass, and so on). A phase shift brought about by an electrical signal traveling a path through particular biological tissue may correspond to a capacitive (or inductive) component of impedance of the biological tissue, for example. In a case where impedance is frequency-dependant, an electrical signal having one frequency (or one set of frequencies) may follow a path through biological tissue different from a path followed by an electrical signal having another frequency (or another set of frequencies).

Biological elements may respond to different signals in different ways. For example, a pulse of a signal may activate an action potential of nerve fibers in muscle tissue if a slope of the pulse is sufficiently steep. On the other hand, if a pulse is not steep enough, then the same nerve fibers may accommodate (e.g., “adjust”) to current flow of the pulse so that no action potential is activated. This illustrates an example where applied signals may affect biological elements for which the signals are used to diagnose. For another such example, a 10,000 Hz sinusoidal signal applied to muscle tissue may increase permeability of the muscle tissue. Accordingly, application of particular signals may affect muscle tissue so that resistance of the muscle tissue changes in response to the applied signals. Different applied signals (e.g., different by frequency, waveshape, voltage level, and so on) may affect particular biological elements differently. Thus, for example, different applied signals may give rise to different resistances of a particular biological element.

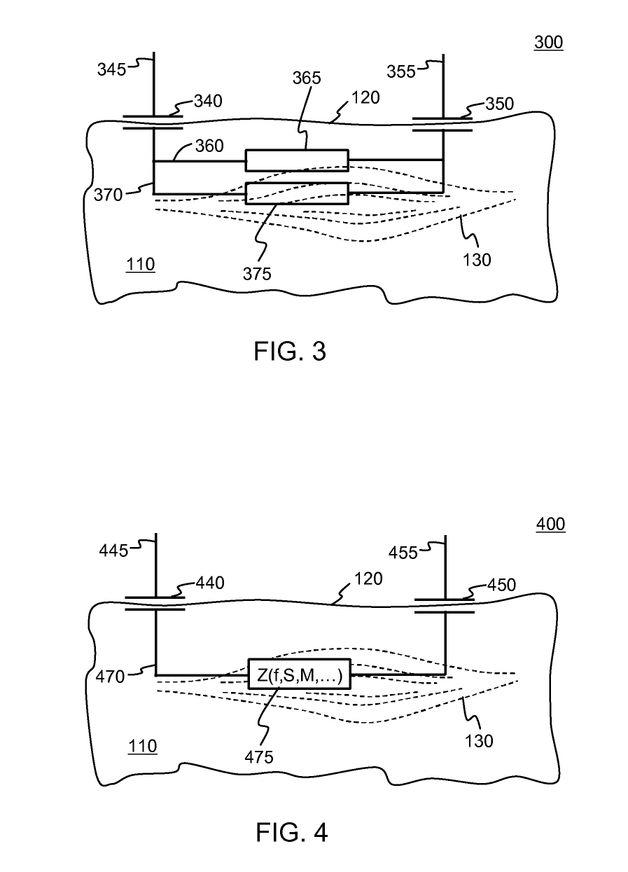

FIG. 3 is a cross-sectional schematic diagram illustrating an electrical component analogue corresponding to biological elements of a subject, such as those introduced in FIG. 1, according to an embodiment 300. Where two low-resistance regions are separated by a high-resistance region, i.e. a near-insulator, a capacitor may be formed and capacitive effects (e.g., phase shift, impedance, and so on) may occur. Thus, where an electrode (e.g., 140 and 150) is separated from nerve or muscle in underlying tissue by skin (more specifically, the stratum corneum), there may be a capacitor. Accordingly, electrode 140 may be represented by capacitor 340 and electrode 150 may be represented by capacitor 350. Capacitors may impede flow of current of an electronic signal, but an extent of such impedance may depend, at least in part, on pulse duration or frequency of the electronic signal. For direct current (e.g., unidirectional current, 0 Hz) or long-duration slowly-varying pulses of current, skin impedance may be relatively high and electrical energy may be (mostly) dissipated in the stratum corneum. For short bursts of current, capacitive impedance of the stratum corneum may be low and electrical energy may be (mostly) dissipated in underlying tissues. Membranes of cells of various biological elements may also give rise to capacitance (e.g., two low-resistance regions separated by a high-resistance membrane).

Leads 345 and 355 may carry electrical signals to electrodes represented by capacitors 340 and 350, respectively. Such electrical signals may follow any of a number of paths, such as 360 or 370, as discussed above. For example, path 360 may have one impedance 365 while path 370 may have another impedance 375. Accordingly, in one implementation, an electrical signal having a particular frequency may follow path 360 and another electrical signal having another particular frequency may follow path 370. In another implementation, an electrical signal having a particular waveshape (e.g., sinusoid, sawtooth, triangular, square, pulse width, duty cycle, rise/fall time, slope, and so on) may follow path 360 and another electrical signal having another particular waveshape may follow path 370. Thus, for example, impedance of a path followed by an electrical signal may depend, at least in part, on frequency and/or waveshape of the electrical signal.

FIG. 4 is a cross-sectional schematic diagram illustrating an electrical component analogue corresponding to biological elements of a subject, such as those introduced in FIG. 1, according to another embodiment 400. As explained above, where two low-resistance regions are separated by a high-resistance region, i.e. a near-insulator, a capacitor may be formed and capacitive effects may occur. Accordingly, electrode 140 may be represented by capacitor 440 and electrode 150 may be represented by capacitor 450.

Leads 445 and 455 may carry electrical signals to electrodes represented by capacitors 440 and 450, respectively. Such electrical signals may follow a path 470 having an impedance 475 that is a function of any of a number of variables. For example, impedance 475 is labeled Z(f, S, M, . . . ), where Z comprises impedance that is a function of one or more various parameters of a signal, such as frequency (f), waveshape (S), voltage or current magnitude (M), and so on. Thus, for example, impedance of a path followed by an electrical signal may depend, at least in part, on frequency, waveshape, and/or magnitude of an electrical signal. To illustrate, in one implementation, an electrical signal having a particular frequency may be subject to one particular impedance while another electrical signal having another particular frequency may be subject to another particular impedance. In another implementation, an electrical signal having a particular waveshape (e.g., sinusoid, sawtooth, triangular, square, pulse width, duty cycle, rise/fall time, slope, and so on) may be subject to one particular impedance while another electrical signal having another particular waveshape may be subject to another particular impedance. Though path 470 is schematically shown as a line, it is to be understood that a path followed by a signal may comprise any of a number, sizes, and/or shapes of circuitous directions. For example, path 470 may comprise variable cross-sectional areas at different portions of the path. Some portions of path 470 may comprise one or more various portions of biological elements. In one implementation, path 470 may include a portion of muscle tissue and a portion of a tendon. Such a path 470 may have one particular impedance for a signal having one frequency while path 470 may have another particular impedance for a signal having another frequency. Of course, this is merely one example of many possibilities, and claimed subject matter is not limited in this respect.

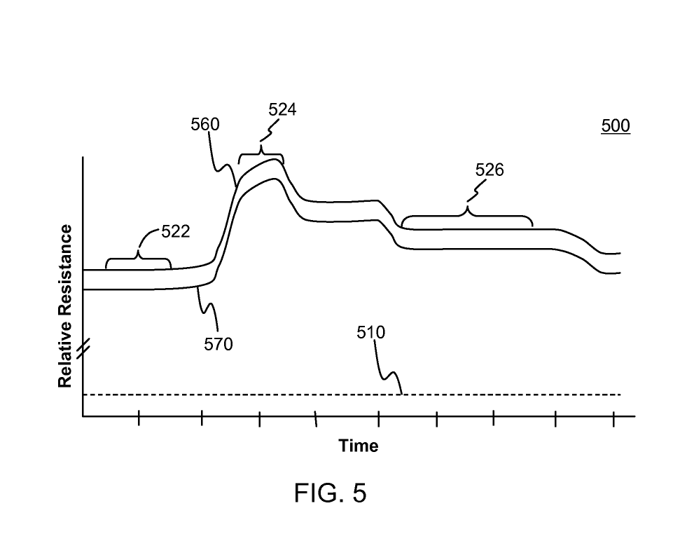

FIG. 5 is a plot 500 of characteristics for relative resistance of biological elements of a subject for two electrical signals as a function of time, according to an embodiment. For example, electrodes 140 and 150 (FIG. 1) may be used to apply first and second electrical signals to a subject. The first and second signals may have frequencies and/or waveshapes different from one another, for example. Accordingly, paths taken by first and second signals may be different from one another so that first and second signals may experience different resistances (e.g., impedances). For example, a first signal may experience a relative resistance shown by curve 560, while a second signal may experience a different relative resistance shown by curve 570. Changing conditions of an electrode-skin interface (e.g., electrodes or skin) may give rise to time-varying relative resistances of first and second signals. Moreover, such changing conditions may affect first and second signals in substantially the same way. For example, even though a frequency of the first signal is different from a frequency of the second signal, changing conditions of electrodes, localized skin conditions, or an electrode-skin interface may affect the first and second signals substantially the same. Thus, for example, magnitude of curve 560 changes with time as does magnitude of curve 570 (e.g., as 560 goes up (or down), so does curve 570). On the other hand, the different signals having different frequencies (and/or waveshapes, and so on) may follow different paths having different resistances. In one implementation, this difference in resistance may be substantially constant over time. In another implementation, this difference may be relatively slowly varying so as to be substantially constant over time spans greater (e.g., by a factor of several or more, just to give an example) than time spans involving changing conditions of electrodes, localized skin conditions, or an electrode-skin interface. Dashed line 510 comprises a plot of a difference between 560 and 570: curves 560 and 570 may be a constant vertical distance apart from each other.

Affects of changing conditions of electrodes, skin, or an electrode-skin interface may be indicated by rise and fall of curves 560 and 570 over time. For example, in region 522, electrodes used to apply the first and second signals may be electrically contacting skin in a relatively desirable fashion: e.g., secure adhesion and relatively good electrical conduction through moist skin. Accordingly, first and second signals may experience relatively low resistance. However, at region 524, the electrodes may have been physically disturbed and may have pulled partially away from the skin, or the skin may have dried up somewhat. Accordingly, first and second signals may experience increased resistance. At region 526, the electrodes may have been physically disturbed again, but may have been pushed against the skin, or the skin may have moistened up somewhat due to perspiration (e.g., in human subjects). Accordingly, first and second signals may experience decreased resistance. In such cases, time-varying relative resistances of first and second signals may be brought about by changing conditions of electrodes or an electrode-skin interface.

In some embodiments, resistance (e.g., impedance) of biological tissue, such as muscle, fascia, and so on, may depend, at least in part, on presence, location, severity, and/or extent of: inflammation; length-tension relationship of the tissue; forces on the tissue; amount of muscle flexion or extension; injuries; muscle atrophy; hot spots; trigger points; amount of blood or inflammatory fluids present; permeability; elasticity of muscle; and/or cell structure; just to name a few examples. Such conditions of biological tissue (or other biological elements, such as bone, cartilage, ligaments, organs, and so on) may determine, at least in part, how efficiently electrical current of a signal applied via electrodes may travel.

For example, a muscle in a particular amount of flexion may have a resistance (e.g., impedance) different from that of the same muscle in extension. Here, the amount of flexion or extension may determine, at least in part, the amount of resistance. In another example, an injured portion of a muscle may have a resistance different from that of a healthy portion of the same muscle. In yet another example, an inflamed muscle may have a resistance different from that of a non-inflamed muscle. In still another example, a muscle subject to stress (e.g., from muscle imbalance) may have a resistance different from that of the same muscle not subject to such stress. Here, the amount of stress may determine, at least in part, the amount of resistance. In yet another example, a joint with injured ligaments may have a resistance different from that of a healthy joint. In yet another example, an organ with cancer cells may have a resistance different from that of a healthy version of the same organ. Here, the amount of cancer cells may determine, at least in part, the amount of resistance. In yet another example, an amount of blood circulation in a portion of a biological element may have a resistance different from that of another amount of blood circulation in the same portion of the biological element.

In the above examples, resistance of a signal may depend, at least in part, on the frequency of the signal. Thus, to revisit some of the examples above, the resistance of a muscle in a particular amount of flexion for a first signal having a first frequency may be different from that of a second signal having a second frequency. In another example, the resistance of a muscle in a particular amount of extension for a first signal having a first frequency may be different from that of a second signal having a second frequency. In still another example, the resistance of an injured portion of muscle for a first signal having a first frequency may be different from that of a second signal having a second frequency. In still another example, the resistance of an inflamed muscle for a first signal having a first frequency may be different from that of a second signal having a second frequency. In yet another example, the resistance of a muscle subject to a particular amount of stress for a first signal having a first frequency may be different from that of a second signal having a second frequency. In yet another example, the resistance of an organ with cancer cells for a first signal having a first frequency may be different from that of a second signal having a second frequency. In yet another example, the resistance of a biological element having a particular amount of blood circulation for a first signal having a first frequency may be different from that of a second signal having a second frequency.

In the above examples, resistance of a signal may depend, at least in part, on waveshape of the signal. Thus, to revisit some of the examples above, the resistance of a muscle in a particular amount of flexion for a first signal having a first waveshape may be different from that of a second signal having a second waveshape. In another example, the resistance of a muscle in a particular amount of extension for a first signal having a first waveshape may be different from that of a second signal having a second waveshape. In still another example, the resistance of an injured portion of muscle for a first signal having a first waveshape may be different from that of a second signal having a second waveshape. In still another example, the resistance of an inflamed muscle for a first signal having a first waveshape may be different from that of a second signal having a second waveshape. In yet another example, the resistance of a muscle subject to a particular amount of stress for a first signal having a first waveshape may be different from that of a second signal having a second waveshape. In yet another example, the resistance of an organ with cancer cells for a first signal having a first waveshape may be different from that of a second signal having a second waveshape. In yet another example, the resistance of a biological element having a particular amount of blood circulation for a first signal having a first waveshape may be different from that of a second signal having a second waveshape.

In a particular implementation, a frequency of a first signal may be different from a frequency of a second signal by any amount, which may be selected based, at least in part, on the biological elements being analyzed or tested, for example. In some implementations, a combination of frequencies for a first and second signal may be selected so that a difference in resistance of the skin for the first and second signal is negligible while a difference in resistance of some biological element under test for the first and second signal is not negligible. Such a difference in resistance between two signals having different frequencies may be based, at least in part, on a capacitive nature of skin (e.g., elements 340 or 350), as explained above. In other implementations, difference in intrinsic resistance of skin for a first signal from that of a second signal may be measured a priori to measuring any biological elements to be tested. Intrinsic resistance of skin may comprise resistance of the skin based, at least in part, on properties of the skin (e.g., pH, moisture, or any qualities of skin that vary among subjects or a particular subject from time to time) as opposed to any resistance variations due, at least in part, to skin-electrode interface variations, as described above.

Some particular numerical examples are: a first signal having a frequency of 1,000 Hz and a second signal having a frequency of 1,100 Hz, 10,000 Hz, 20,000 Hz, 100,000 Hz, or 1,000,000 Hz; a first signal having a frequency of 10,000 Hz and a second signal having a frequency of 10,100 Hz, 11,000 Hz, 20,000 Hz, 100,000 Hz, or 1,000,000 Hz; a first signal having a frequency of 100,000 Hz and a second signal having a frequency of 101,000 Hz, 110,000 Hz, 200,000 Hz, or 1,000,000 Hz; and so on. (In cases of non-sinusoidal wave-shapes, such frequencies may comprise first order Fourier frequencies, as explained below, for example.) Of course, these examples are intended to illustrate that any combination of any values of frequencies may be selected, and claimed subject matter is not so limited.

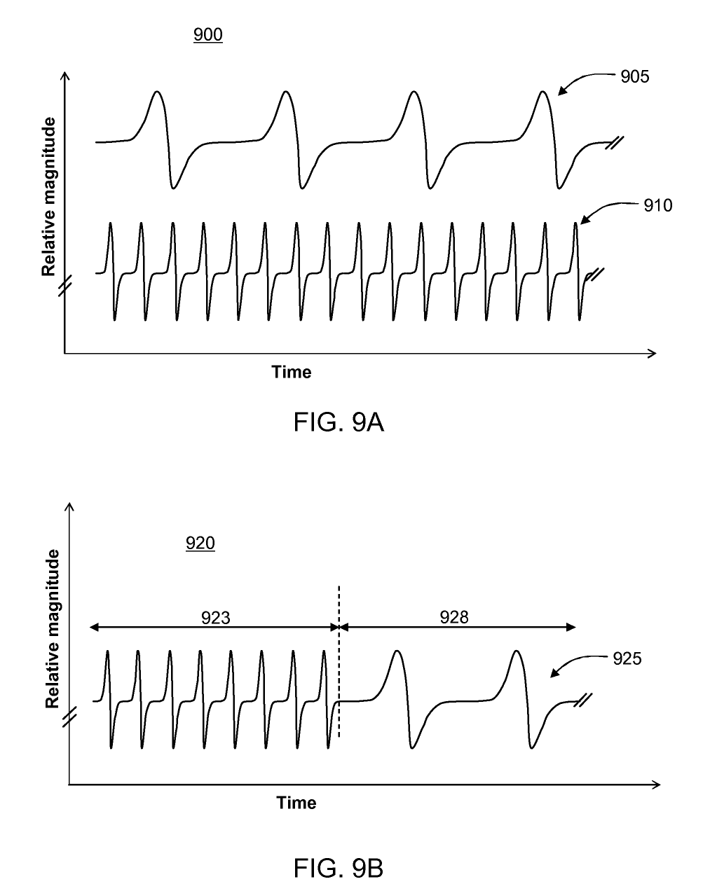

Though examples above recite single frequencies, it is understood that a signal may comprise a Fourier spectrum of frequencies (e.g., for non-perfect sinusoids). In some example implementations, however, a single frequency may refer to a frequency that a cycle of a wave repeats over time. To illustrate, FIG. 9B shows a section of a signal over a span 923. If, for sake of an example, span 923 corresponds to one second, then the frequency of this signal would be 8 Hz (a cycle of the wave occurs 8 times in one second). In contrast, this signal comprises a non-perfect sinusoid so that this signal may comprise a Fourier spectrum of frequencies (of which 8 Hz may be the first order frequency). Accordingly, in one implementation, a frequency of a first electrical signal may be considered to be substantially different from that of a second electrical signal if their first order frequencies are different from each other by one percent or more, for example.

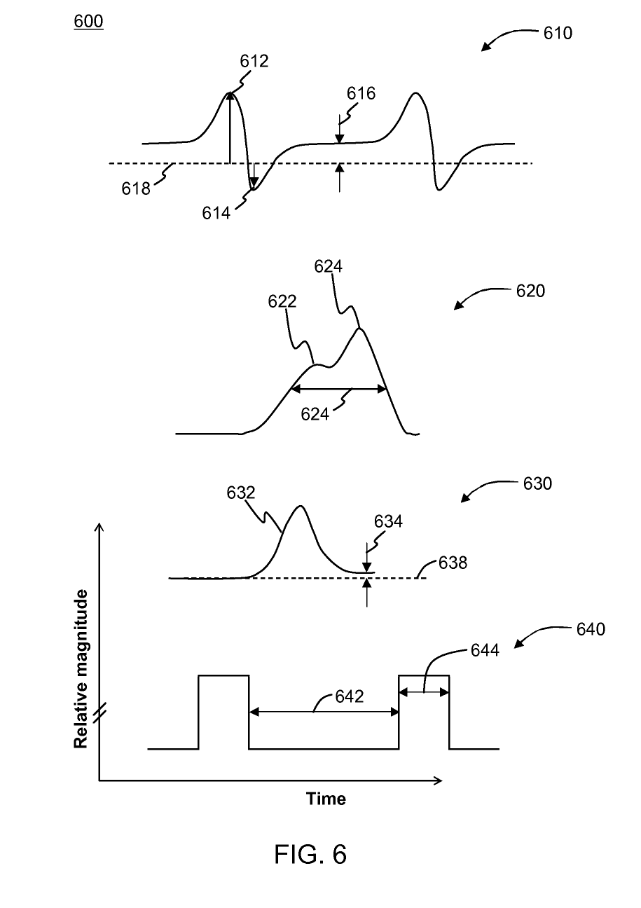

FIG. 6 includes a number of example waves plotted as magnitude of voltage or current versus time, according to an embodiment. For example, a first or second signal applied to a subject via electrodes may comprise any such wave or variation thereof. Of course, there are an endless variety of waves having different shapes or characteristics, and FIG. 6 shows merely a small number of possibilities. Here, FIG. 6 is useful for helping to explain meanings of some terms that are used to describe signal characteristics. Wave 610 includes a positive-going peak magnitude 612 (e.g., curve is concave downward), a negative-going peak magnitude 614 (e.g., curve is concave upward), and an offset 616 from a reference level 618, which may be zero volts or ground, for example. Wave 620 includes a peak magnitude 624 and a feature 622, which may comprise a change in slope, an extra peak or bump, or any type of feature or features superposed onto what may be considered a primary wave (e.g., 620). Wave 620 also has a width 624 (e.g., pulse width), which may be described as full width at half max (FWHM). Wave 630 includes an instantaneous or average slope 632 and a portion having an offset 634 from a reference level 638. Wave 640 shows a square wave having a pulse width 644 and duty cycle that may be described by time 642 between pulses. Of course, any wave may be described by any parameters introduces above, and claimed subject matter is not so limited.

In a particular implementation, a waveshape of a first signal may be different from a waveshape of a second signal by any amount or fashion, which may be selected based, at least in part, on the biological elements being analyzed or tested, for example. In some implementations, a combination of waveshapes for a first and second signal may be selected so that a difference in resistance of the skin for the first and second signal is negligible while a difference in resistance of some biological element under test for the first and second signal is not negligible. Such a difference in resistance between two signals having different waveshapes may be based, at least in part, on a capacitive nature of skin (e.g., elements 340 or 350), as explained above. In other implementations, difference in resistance of skin for a first and a second signal having particular waveshapes may be measured a priori to measuring any biological elements to be tested. Such a difference in resistance between the two signals having different waveshapes may be separate from any resistance variations due, at least in part, to skin-electrode interface variations, as described above.

Some particular examples are: a first signal having a waveshape portion with a first slope (e.g., 632) and a second signal having a corresponding waveshape portion with a second slope; a first signal having a triangular waveshape and a second signal having a sawtooth waveshape; a first signal having a first duty cycle and a second signal having a second duty cycle; a first signal having a first pulse width and a second signal having a second pulse width; and so on. Of course, these examples are intended to illustrate that any combination of waveshapes may be selected, and claimed subject matter is not so limited.

In a particular implementation, intensity values of a first signal may be different from those of a second signal by any amount, which may be selected based, at least in part, on the biological elements being analyzed or tested, for example. Here, the meaning of “intensity values” may include values of voltage or current of any portion of a wave, such as a positive peak (e.g., 612), a negative peak (e.g., 614), an offset (e.g., 616) of a wave from a reference (e.g., ground), and so on.

In some implementations, a combination of intensity values for a first and second signal may be selected so that a difference in resistance of the skin for the first and second signal is negligible while a difference in resistance of some biological element under test for the first and second signal is not negligible. Such a difference in resistance between two signals having different intensity values may be based, at least in part, on a capacitive nature of skin (e.g., elements 340 or 350), as explained above. In other implementations, difference in resistance of skin for a first and a second signal having particular intensity values may be measured a priori to measuring any biological elements to be tested. Such a difference in resistance between the two signals having different intensity values may be separate from any resistance variations due, at least in part, to skin-electrode interface variations, as described above.

Some particular examples are: a first signal having a first peak value and a second signal having a second peak value; a first signal having a first offset and a second signal having a second offset; a first signal having a first average value and a second signal having a second average value; and so on. Of course, these examples are intended to illustrate that any combination of intensity values may be selected, and claimed subject matter is not so limited.

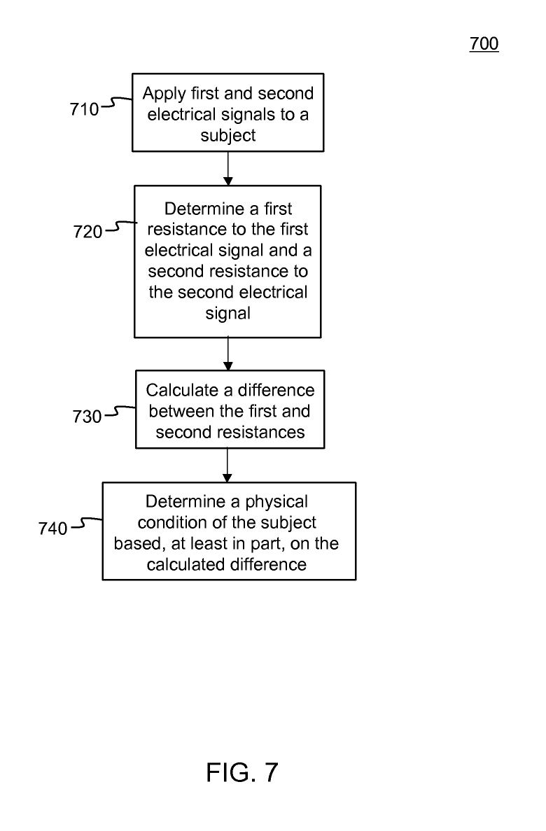

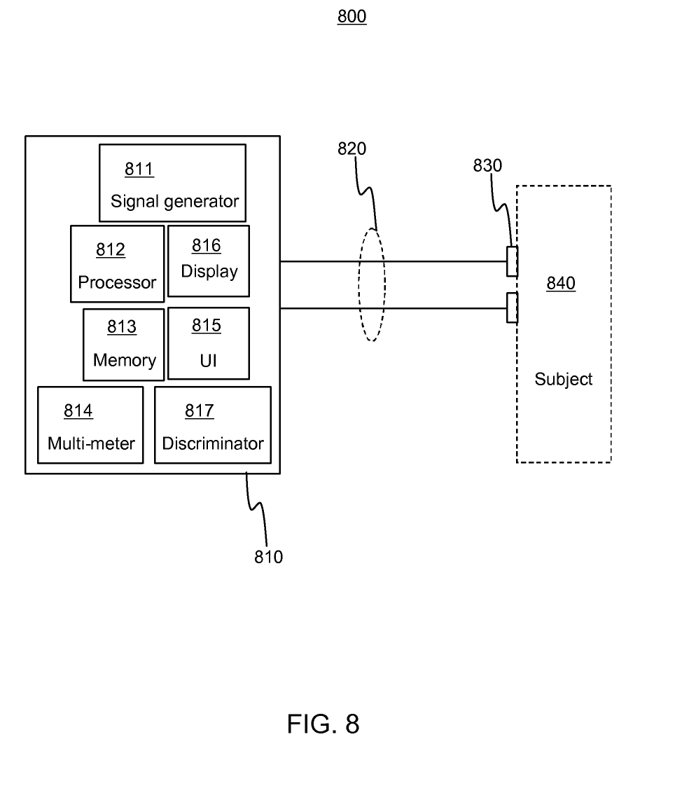

FIG. 7 is a flow diagram of a process 700 for determining a physical condition of a subject, according to an embodiment. At block 710, a first electrical signal may be applied to a subject via electrodes and a second electrical signal may be applied to the subject via the same electrodes as those of the first signal. At block 720, a first resistance of the first signal may be determined and a second resistance of the second signal may be determined. For example, using Ohm's Law resistance may be determined by measuring current resulting, at least in part, from an applied voltage of a signal. For another example, using Ohm's Law resistance may be determined by measuring voltage resulting, at least in part, from an applied current of a signal. At block 730, a difference between first and second resistances may be determined or calculated. Such a difference may comprise a phase shift and/or resistance. For a particular numerical example, a determined resistance of a first signal may comprise a shift in phase of current from that of voltage of the applied first signal of 10 degrees and a resistance of 2500 ohms, and a determined resistance of a second signal may comprise a shift in phase of current from that of voltage of the applied second signal of 8 degrees and a resistance of 2200 ohms, so that a difference may comprise a 2 degree phase shift and 300 ohms.

At block 740, a physical condition of a subject may be determined based, at least in part, on the difference in resistance between that of the first signal and that of the second signal. Of course, such details of process 700 are merely examples, and claimed subject matter is not so limited.

FIG. 8 is a schematic diagram illustrating a system 800 for performing a process, such as 700 for example, for determining a physical condition of a subject, according to an embodiment. For example, system 800 may comprise a device 810, cables 820, and electrodes 830. Device 810 may generate one or more signals that may be applied to a subject 840 via electrodes 830. Device 810 may include a signal generator 811 to generate signals having any of a number of parameters, such as waveshape, magnitude, frequency, offset (e.g., from zero volts), and so on. Signal generator 811 may generate more than one signal at a time (e.g., as shown in plot 900 below), or may repeatedly and alternately generate a first signal and a second signal (e.g., as shown in plot 920 below).

For example, FIGS. 9A and 9B are plots of characteristics for first and second electrical signals as a function of time, according to an embodiment. Plot 900 of FIG. 9A includes a first signal 905 simultaneously present with a second signal 910. First signal 905 may have a different frequency, waveshape, and/or magnitude than that of second signal 910. A composite signal (not shown) may comprise a summation of first and second signals 905 and 910 (e.g., magnitudes of signals 905 and 910 added together). For example, signals 905 and 910 simultaneously present in a single current path may be superposed to form a composite signal.

Plot 920 of FIG. 9B includes a first signal 923 repeatedly alternating with a second signal 928. First signal 923 may have a different frequency, waveshape, and/or magnitude than that of second signal 928. For a particular example, first signal 923 may be provided to a subject for one second, followed by second signal 928 for one second, followed by the first signal 923 for one second, followed by the second signal 928 for one second, and so on. Time spans for 923 and 928 (e.g., one second in the example above) are desirably substantially less than (e.g., an order of magnitude less than) time spans corresponding to motions of a subject or changes in adhesive states of electrodes on skin, for example. Thus, it may be desirable for system 800 to “sample” resistances at a rate faster than electrode-skin interface resistance changes. Such changes may result from subject motion, changes of pressure on electrodes, or weakening electrode adhesive, just to name a few examples. For instance, sample time (e.g., time spans of 923 and 928) of about a tenth of a second may be adequate for accurate measurements considering that subject movements may be of the order of several seconds (e.g., repetitive exercises performed by the subject may occur in two second cycles).

Returning to FIG. 8, a processor 812 may be used to calculate or determine resistance to a signal provided to electrodes 830, which may be electrically connected to subject 840. Processor 812 may perform such calculations or determinations using parameters measured by multi-meter 814. Such parameters may include voltage, current, phase shift, and so on.

A discriminator 817 may decompose or separate a composite signal into two or more individual signals. For example, a first voltage signal and a second voltage signal may be provided to electrodes 830 simultaneously so that the electrodes apply a composite voltage signal to subject 840. Such a composite voltage signal may include a superposition of the first and second voltage signals. Current of the composite voltage signal flowing through subject 840 may be decomposed by discriminator 817 so that the current is separated into a first current signal and a second current signal, which may be measured by multi-meter 814, for example. In one implementation, discriminator 817 may comprise one or more frequency filters (e.g., low-pass, high-pass, or notch filters, and so on) to perform such signal separation. In another implementation, discriminator 817 may comprise one or more amplitude filters (e.g., involving resistor networks, diodes, etc.) to perform such signal separation. In yet another implementation, discriminator 817 may comprise one or more waveshape filters to perform such signal separation. In any case, a composite signal provided to discriminator 817 (e.g., by cables 820) may comprise a digital signal. Here, an analog to digital converter (not shown) may be used to convert an analog composite signal flowing through subject 840 to a digital composite signal. Software executed by processor 812 may be used to identify or distinguish one waveform of one signal from another waveform of another signal in a digital composite signal. With information from such a processor, discriminator 817 may separate the separate waveforms and multi-meter 814 may then measure current or voltage of the separated waveforms.

Device 810 may further include memory 813 to store values of parameters measured by multi-meter 814, or generated by processor 812 or discriminator 817, for example. A user interface 815 may include a keypad or touchscreen by which a user may provide operational instructions to device 810. A display 816 may display any information to a user. Of course, such details of system 800 are merely examples, and claimed subject matter is not so limited.

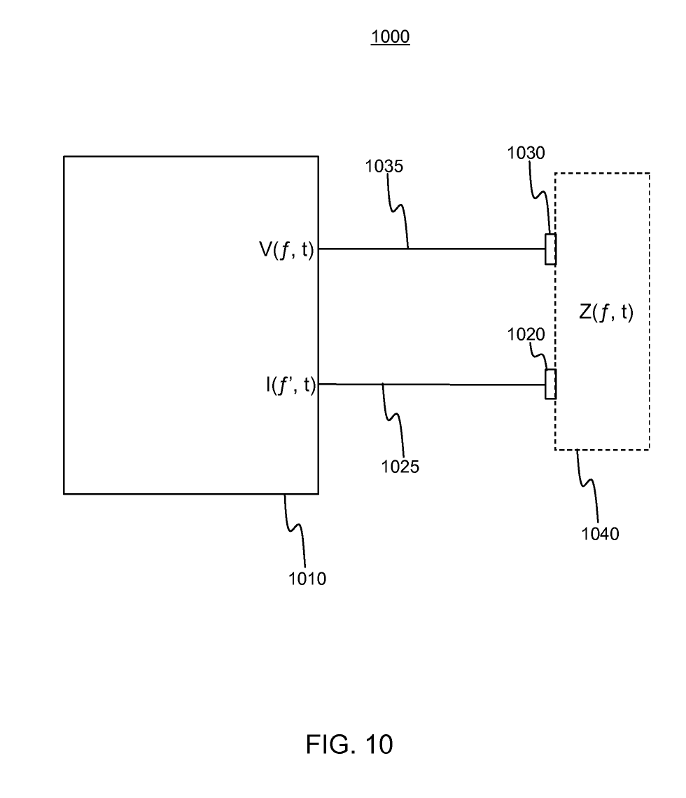

FIG. 10 is a schematic block diagram illustrating a system 1000, which may be similar to the example embodiment system 800 shown in FIG. 8. For example, device 1010 may include components shown in device 810, such as 811, 812, 813, 814, 815, 816, and 817, though claimed subject matter is not so limited. Device 1010 may generate one or more time-dependent signals represented by V(f, t), where f represents frequency and t represents time. Such time dependence may involve cyclically varying wave functions, for example. Such signals may be applied to a subject 1040 via electrode 1030 and lead 1035. Subject 1040 may present an impedance Z(f, t) to current I(f′, t) imparted by V(f, t), where distinctions between f and f′ are explained below. It is understood that electrical signal flow may be bi-directional, such as cases where polarity reverses cyclically (e.g., alternating current). Accordingly, even though embodiments may be described as having an output or an input, such designations may be reversed. For example, current I(f′, t) may be provided on lead 1035 and V(f, t) may be on lead 1025 via electrode 1020, or vise versa. Claimed subject matter is not limited in this respect.

Z(f, t), as indicated by inclusion of the variable for time, t, may be time-dependent. Such time-dependence may account for variable resistance of electrode-skin interface over time (e.g., of the order of one or two seconds, minutes, or hours). Z(f, t) may include impedances of electrode-skin interfaces, and impedances of one or more biological elements of subject 1040, for example.

In the example embodiment, V(f, t) may comprise a composite voltage including voltages of two signals: signal 1 and signal 2. These signals may individually include one or more frequency components. For example, in the case of signal 1 comprising a mathematically perfect sine wave having a frequency f₁ and signal 2 comprising a mathematically perfect sine wave having a frequency f₂, V(f, t) may comprise signal 1 with exactly one frequency component and signal 2 with exactly one frequency component, written as

V(f,t)=V₁(f₁,t)+V₂(f₂,t) Eqn. (1)

On the other hand, or in a more general case, signal 1 and/or signal 2 may comprise non-sinusoidal waves (e.g., square wave, ramp, double-exponential wave, wave-shape with duty cycle, and so on). In such cases, Fourier components of these waves may comprise a series or sum of frequency terms. For example, signal 1 may have frequency terms fᵢⱼ and signal 2 may have frequency terms f₂ₖ, where j, k=1, 2, 3, . . . Accordingly, V(f, t) comprising signal 1 and signal 2 with such frequency components, may be written as

V(f,t)=Σ[V₁(f₁ⱼ,t)+V₂(f₂ₖ,t)], Eqn. (2)

where

ΣV₁(f₁ⱼ,t)=V(f₁₁,t)+V(f₁₂,t)+V(f₁₃,t)+V(f₁₄, t)+ . . . Eqn. (3)

and

ΣV₂(f₂ⱼ,t)=V(f₂₁,t)+V(f₂₂,t)+V(f₂₃,t)+V(f₂₄,t)+ . . . Eqn. (4)

Subject 1040 may have an impedance Z(f, t), which is written in bold-face to represent the fact that this impedance may comprise two or more components. For example, Z(f, t) may be written as

Z(f,t)=[Z(f₁ⱼ,t),Z(f₂ₖ,t)], for j, k=1, 2, 3, . . . Eqn. (5)

where

Z(f₁ⱼ,t)=Z(f₁₁,t),Z(f₁₂,t),Z(f₁₃,t),Z(f₁₄,t), . . . Eqn. (6)

and

Z(f₂ⱼ,t)=Z(f₂₁,t),Z(f₂₂,t),Z(f₂₃,t),Z(f₂₄,t), . . . Eqn. (7)

Here, Z(f₁ⱼ, t) may represent a set of impedances for signal 1 and Z(f₂ⱼ, t) may represent a set of impedances for signal 2. For example, as discussed above, impedances of various biological elements of subject 1040 may be frequency-dependent. Accordingly, impedance terms for individual frequencies may correspond to individual voltage terms of the same individual frequencies. In one implementation, difference of capacitive reactances (e.g., the imaginary component of impedance) of electrode-skin interfaces between that of signal 1 and that of signal 2 may be negligible and ignored in some applications. For example, capacitive reactances of electrode-skin interfaces for signal 1 and for signal 2 may be substantially equal or similar (e.g., different by less than about a few percent) for some ranges of frequencies. For a numerical example, capacitive reactance of electrode-skin interfaces (e.g., at 1020 and 1030) for signal 1 having a first order frequency (e.g., a largest term in a Fourier series of terms) of 10,000 Hz may be less than 2% different from a capacitive reactance for signal 2 having a first order frequency of 12,000 Hz. Thus, it may be desirable to discount capacitive reactance of electrode-skin interfaces so that any remaining capacitive reactances may be based, at least in part, on biological elements (e.g., sans skin) of a subject.

In another implementation, difference of capacitive reactances of electrode-skin interfaces between that of signal 1 and that of signal 2 may be accounted for by maintaining a table of values (or other format of such information) of capacitive reactances of electrode-skin interfaces for a plurality of frequencies for particular subjects or for types of subjects. For example, subjects may have particular skin conditions (e.g., having various values of pH, moisture content, and so on). For example, for a particular subject (or particular class of subjects), a table of values may comprise values for capacitive reactance of electrode-skin interfaces for different signal frequencies. Thus, it may be desirable to discount capacitive reactance of electrode-skin interfaces so that any remaining capacitive reactances may be based, at least in part, on biological elements (e.g., sans skin) of a subject.

In the example embodiment, I(f′, t) may comprise a composite current including currents of signal 1 and signal 2. Fourier components of these signals may comprise a series or sum of frequency terms. For example, signal 1 may have frequency terms f′₁ⱼ and signal 2 may have frequency terms f′₂ₖ, where j, k=1, 2, 3, . . . Accordingly, I(f′, t) comprising currents of signal 1 and signal 2 with such frequency components, may be written as

I(f′,t)=Σ[I₁(f′₁ⱼ,t)+I₂(f′₂ₖ,t)] Eqn. (8)

where

ΣI₁(f′₁ⱼ,t)=I(f′₁₁,t)+I(f′₁₂,t)+I(f′₁₃,t)+I(f′₁₄,t)+ . . . Eqn. (9)

and

ΣI₂(f′₂ⱼ,t)=I(f′₂₁,t)+I(f′₂₂,t)+I(f′₂₃,t)+I(f′₂₄,t)+ . . . Eqn. (10)

Fourier components of these current waves may comprise a series or sum of frequency terms that may be different from corresponding terms in ΣV₁(f₁ⱼ, t) and ΣV₂(f₂ⱼ, t). For example, impedances of subject 1040 may shift the phases of currents of the different frequencies with respect to the phases of the corresponding voltages. Thus, frequencies of some Fourier terms of current may be altered from those of the voltage based, at least in part, on frequency-dependent impedances (e.g., capacitive or inductive reactances). (In other words, a shape of a current wave may be distorted from that of the voltage wave by frequency-dependent impedances: Thus, frequencies of Fourier terms of the current may be different from those of the voltage to account for the wave-shape distortion. If impedance were not frequency-dependent, for example, then there may be no phase shifts between voltage and current Fourier terms.)

Using Ohm's Law for signal 1,

Z(f₁ₘ,t)=V(f₁ₘ,t)/I(f′₁ₘ,t), for m=1, 2, 3, . . . Eqn. (11)

and for signal 2,

Z(f₂ₘ,t)=V(f₂ₘ,t)/I(f′₂ₘ,t), for m=1, 2, 3, . . . Eqn. (12)

A difference between impedances for signal 1 and for signal 2 may then be written as

ΔZ=∥Σ[Z(f₁ₘ,t)−Z(f₂ₘ,t)]∥ Eqn. (13)

An example of ΔZ may be similar to 510 shown in FIG. 5. Of course, such details of voltage, current, and impedance are merely examples, and claimed subject matter is not so limited.

It will, of course, be understood that, although particular embodiments have just been described, claimed subject matter is not limited in scope to a particular embodiment or implementation. For example, one embodiment may be in hardware, such as implemented on a device or combination of devices, for example. Likewise, although claimed subject matter is not limited in scope in this respect, one embodiment may comprise one or more articles, such as a storage medium or storage media that may have stored thereon instructions capable of being executed by a specific or special purpose system or apparatus, for example, to lead to performance of an embodiment of a method in accordance with claimed subject matter, such as one of the embodiments previously described, for example. However, claimed subject matter is, of course, not limited to one of the embodiments described necessarily. Furthermore, a specific or special purpose computing platform may include one or more processing units or processors, one or more input/output devices, such as a display, a keyboard or a mouse, or one or more memories, such as static random access memory, dynamic random access memory, flash memory, or a hard drive, although, again, claimed subject matter is not limited in scope to this example.

The terms, “and” and “or” as used herein may include a variety of meanings that will depend at least in part upon the context in which it is used. Typically, “or” if used to associate a list, such as A, B or C, is intended to mean A, B, and C, here used in the inclusive sense, as well as A, B or C, here used in the exclusive sense. Embodiments described herein may include machines, devices, engines, or apparatuses that operate using digital signals. Such signals may comprise electronic signals, optical signals, electromagnetic signals, or any form of energy that provides information between locations.

In the preceding description, various aspects of claimed subject matter have been described. For purposes of explanation, specific numbers, systems, or configurations may have been set forth to provide a thorough understanding of claimed subject matter. However, it should be apparent to one skilled in the art having the benefit of this disclosure that claimed subject matter may be practiced without those specific details. In other instances, features that would be understood by one of ordinary skill were omitted or simplified so as not to obscure claimed subject matter.

While there has been illustrated and described what are presently considered to be example embodiments, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular embodiments disclosed, but that such claimed subject matter may also include all embodiments falling within the scope of the appended claims, and equivalents thereof.

What is claimed is:

1. A method comprising:
applying a first electrical signal to particular locations of a subject via at least one electrode pair;
applying a second electrical signal to said particular locations of said subject via said at least one electrode pair, wherein said second electrical signal is applied alternately and repeatedly with said first electrical signal;
determining a first electrical impedance based, at least in part, on said first electrical signal;
determining a second electrical impedance based, at least in part, on said second electrical signal; and
removing at least a portion of varying electrical characteristics of said at least one electrode pair by calculating a difference between said first electrical impedance and said second electrical impedance.

2. The method of claim 1, further comprising determining a physical condition of said subject based, at least in part, on said difference.

3. The method of claim 2, wherein said physical condition of said subject comprises a muscle condition of said subject.

4. The method of claim 1, wherein a frequency of said first electrical signal is substantially different from a frequency of said second electrical signal.

5. The method of claim 1, wherein a waveshape of said first electrical signal is substantially different from a waveshape of said second electrical signal.

6. The method of claim 1, wherein a peak voltage of said first electrical signal is substantially the same as a peak voltage of said second electrical signal.

7. The method of claim 1, wherein said first electrical signal and said second electrical signal are applied to said subject while said subject is substantially moving.

8. The method of claim 1, wherein said first electrical signal and said second electrical signal are applied to said subject while said at least one electrode is submerged in a liquid.

9. The method of claim 1, further comprising:
if said difference between said first electrical impedance and said second electrical impedance is less than a particular threshold, then:
changing the frequency of said first electrical signal or said second electrical signal so as to increase said difference.

10. The method of claim 1, wherein said varying electrical characteristics comprise electrode-skin interface resistance.

11. A method comprising:
applying a composite electrical signal to particular locations of a subject via at least one electrode pair, wherein said composite signal comprises a first electrical signal and a second electrical signal;
determining a first electrical impedance based, at least in part, on said first electrical signal;
determining a second electrical impedance based, at least in part, on said second electrical signal; and
removing at least a portion of varying electrical characteristics of said at least one electrode pair by calculating a difference between said first electrical impedance and said second electrical impedance.

12. The method of claim 11, further comprising:
determining a physical condition of said subject based, at least in part, on said difference between said first electrical impedance and said second electrical impedance.

13. The method of claim 11, wherein said determining said first and second electrical impedances further comprises:
measuring a composite electrical current responsive, at least in part, to said composite electrical signal and electrical properties of said subject;
decomposing said composite electrical current to determine a first current and a second current responsive, at least in part, to said first and second electrical signals, respectively; and
determining said first and second electrical impedances based, at least in part, on said first and second currents, respectively.

14. The method of claim 11, wherein a frequency of said first electrical signal is substantially different from a frequency of said second electrical signal.

15. The method of claim 11, wherein said first electrical signal and said second electrical signal are applied to said subject while said subject is substantially moving.

16. The method of claim 11, wherein said varying electrical characteristics of said at least one electrode pair comprises electrical impedance between skin of said subject and said at least one electrode pair.

17. An apparatus comprising:
a port to provide an output signal to a subject via at least one electrode pair; and
a circuit to:
generate said output signal comprising a first electrical signal and a second electrical signal;
determine a first electrical impedance based, at least in part, on said first electrical signal;
determine a second electrical impedance based, at least in part, on said second electrical signal; and
remove at least a portion of varying electrical characteristics of said at least one electrode pair by calculating a difference between said first electrical impedance and said second electrical impedance.

18. The apparatus of claim 17, wherein said first electrical signal and said second electrical signal are simultaneously present in said output signal.

19. The apparatus of claim 18, wherein said circuit is further adapted to
measure a composite electrical current responsive, at least in part, to said output signal and electrical properties of said subject;
decompose said composite electrical current to determine a first current and a second current responsive, at least in part, to said first and second electrical signals, respectively; and
determine said first and second electrical impedances based, at least in part, on said first and second currents, respectively.

20. The apparatus of claim 17, wherein said first electrical signal and said second electrical signal repeatedly alternate between one another in said output signal.
